(19) United States
(12) Patent Application Publication
(10) Pub. No.: US 2006/0270985 A1
(43) Pub. Date: Nov. 30, 2006

(54) Device for administering an injectable product

(76) Inventors: Edgar Hommann, Grossaffoltern (CH); Benjamin Scherer, Uster (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/417,519

(22) Filed: May 4, 2006

Related U.S. Application Data
(63) Continuation of application No. PCT/CH04/00653, filed on Oct. 29, 2004.

(30) Foreign Application Priority Data
Nov. 5, 2003 (DE) 103 51 597.6
Nov. 5, 2003 (DE) 103 51 596.8

(51) Int. Cl. A61M 5/20

(52) U.S. Cl. 604/136

(57) ABSTRACT

A device for administering an injectable product includes a housing, a reservoir for the product, a plunger axially displaceable in a forward-drive direction in the reservoir which effects a dispensing stroke in the forward-drive direction to dispense the product, and a priming mechanism, displaceable in the forward-drive direction and acting on the plunger to permit a priming stroke in the forward-drive direction to remove air from the reservoir. In one embodiment, the device is an auto-injector wherein a dispensing spring acts on the plunger rod in the forward drive direction, the rod being locked in a retaining position in a releasable retaining engagement against the force of the dispensing spring, and wherein a dose metering element is disposed on the housing so it can be displaced to set a product dose and is coupled with the plunger rod by a dose metering engagement so that a dose metering movement of the dose metering element causes an axial displacement of the plunger rod or a stop for the plunger rod when the retaining engagement is in effect.

### CROSS-REFERENCE TO RELATED APPLICATION(S)

This application is a continuation of International Application No. PCT/CH2004/000653, filed on Oct. 29, 2004, which claims priority to German Application No. DE 103 51 597.6, filed on Nov. 5, 2003, and German Application No. DE 103 51 596.8, filed on Nov. 5, 2003, the entire contents of all of which are incorporated herein by reference.

### BACKGROUND

The present invention relates to devices for administering, injecting, delivering or dispensing substances, and to methods of making and using such devices. More particularly, it relates to a device for administering an injectable product, wherein the device may be an injection device such as an auto-injector, and to the process of removing air from a reservoir of the device filled with the product, which process may be referred to as priming.

When administering injections, it is necessary to ensure that no air is injected with the product to be injected. This applies in particular to intravenous injections. To prevent air from being injected, the reservoir and the adjoining components delivering the product upstream are primed. In injection devices in which the product is driven by means of a plunger and a plunger rod acting on the plunger, priming is effected by means of a short priming stroke of the plunger rod and the plunger. The plunger rod constitutes the priming mechanism or at least the output element of a priming mechanism. To avoid unnecessarily dispensing a large amount of product during priming, the priming stroke of the plunger is pre-set beforehand. The priming mechanisms designed for this purpose are generally complex and of a filigree design.

The process of priming automatic injection devices whereby the user does not operate the plunger rod for dispensing product but merely has to trigger the dispensing stroke, i.e., the plunger rod is moved by means of an integrated drive mechanism, is difficult. This applies in particular to auto-injectors for fully automated injection devices, by means of which the injection takes place on a fully automatic basis by triggering the injector after it is positioned on or at a desired site for administering the product. If the auto-injector has an injection cannula as an infusing part, an injection stroke of the injection cannula is also automatically triggered on triggering.

U.S. Pat. No. 4,031,893 describes an auto-injector with a product container, in which a plunger is accommodated and can be moved in the forward drive direction to dispense the product. The plunger is pushed in the forward drive direction by means of a plunger rod. The plunger rod is moved in the forward drive direction due to the force applied by means of a dispensing spring and is locked in a retained position in a releasable retaining engagement against the force of the spring. When the retaining engagement is released, the plunger rod pushes against the plunger. Due to the static friction of the plunger, the plunger is moved in the forward drive direction together with the product container during a first phase of the injection, thereby piercing an injection cannula through the tissue as a result. Once the static friction of the plunger has been overcome, the plunger rod pushes the plunger in the forward drive direction in the container until the container has been completely emptied. To enable different quantities of product to be administered with each plunger rod stroke while using the same size container, an adapter is used which connects the plunger rod to the plunger when the container is only partially full. When the container is completely full, the plunger rod is connected to the plunger directly.

DE 198 22 031 B 1 discloses another auto-injector, which has a sequence control system for separating the injection movement of the injection cannula and the dispensing movement of the plunger. On the one hand, a plunger rod again acts on a plunger accommodated in a product container which can be moved in a forward drive direction and, on the other hand, a drive body separate from the plunger rod acts on the container in the forward drive direction. A drive spring acts on the drive body, which is again locked in a retained position in a retaining engagement. Once the retaining engagement is released, the drive body moves in the forward drive direction under the force of the spring and thus pushes the container with the injection cannula attached to it in the forward drive direction so that the injection cannula pierces the tissue. This injection movement is restricted by means of a stop. Once the stop is reached, the coupling between the drive body and the container is released, so that the spring moves the drive body farther in the forward drive direction without the container. During the course of this movement, it comes into driving contact with the plunger rod and pushes the plunger rod and hence the plunger in the forward drive direction so that the product is dispensed. The container is respectively emptied with each dispensing stroke.

### SUMMARY

In one embodiment, the present invention comprises a device for administering an injectable product with a simple priming mechanism, wherein the priming mechanism is suitable for semi-automatic and fully automatic injection devices, and wherein the device for administering is adapted to dispense or administer a quantity of product which can be selected or adapted to specific individual requirements.

In one embodiment, the present invention comprises a device for administering an injectable product includes a housing, a reservoir for the product, a plunger axially displaceable in a forward-drive direction in the reservoir which effects a dispensing stroke in the forward-drive direction to dispense the product, and a priming mechanism, displaceable in the forward-drive direction and acting on the plunger to permit a priming stroke in the forward-drive direction to remove air from the reservoir. In one embodiment, the device is an auto-injector wherein a dispensing spring acts on the plunger rod in the forward drive direction, the rod being locked in a retaining position in a releasable retaining engagement against the force of the dispensing spring, and wherein a dose metering element is disposed on the housing so it can be displaced to set a product dose and is coupled with the plunger rod by a dose metering engagement so that a dose metering movement of the dose metering element causes an axial displacement of the plunger rod or a stop for the plunger rod when the retaining engagement is in effect.

In one embodiment, the present invention comprises a device for administering an injectable product comprising a housing, a reservoir for the product, a plunger axially displaceable in a forward-drive direction in the reservoir which effects a dispensing stroke in the forward-drive direction in order to dispense a product, and a priming mechanism, which is displaceable in the forward-drive direction and acts on the plunger to permit a priming stroke in the forward-drive direction in order to remove air from the reservoir. The priming mechanism can be moved in the forward-drive direction relative to the plunger rod for the priming stroke. An auto-injector comprises a housing, a product container, a plunger which is axially displaceable in a forward drive direction in the product container, a plunger rod which effects a dispensing stroke in the forward drive direction in order to dispense a product, and a dispensing spring acting on the plunger rod in the forward drive direction. The plunger rod is locked in a retaining position in a releasable retaining engagement against the force of the dispensing spring. A dose metering element is disposed on the housing so that it can be displaced in order to set a product dose and is coupled with the plunger rod by means of a dose metering engagement so that a dose metering movement of the dose metering element causes an axial displacement of the plunger rod or a stop for the plunger rod when the retaining engagement prevails.

In one embodiment, a device for administering an injectable product of the type to which the invention relates comprises a housing, a reservoir for the product, a plunger, a plunger rod and a priming mechanism. The housing may directly constitute the reservoir. However, the reservoir may be a container accommodated by the housing. The plunger is mounted so as to be axially displaceable in the reservoir in the forward-drive direction and product is dispensed from the reservoir by a movement in the forward-drive direction. The plunger rod is mounted so as t be displaceable in the forward-drive direction in order to effect a dispensing stroke, whereby it acts on the plunger in the forward-drive direction and moves the plunger in the forward-drive direction. The plunger rod may be fixedly secured to the plunger axially, for example by means of a screw or bayonet connection or a simple catch connection. In some preferred embodiments, however, it acts on the plunger solely by contact pressure and there is no connection to the plunger other than the contact pressure which occurs during a dispensing stroke. With such a device, a product dose to be dispensed with a dispensing stroke can be set without changing the length of the dispensing stroke. The priming mechanism is used to remove air from the reservoir and the adjoining components of the device carrying the product, disposed upstream. The priming mechanism is also mounted so as to be displaceable. It may effect a priming stroke, during which it acts on the plunger in the forward-drive direction in order to force out any air which may possibly be present in the product-carrying system.

In one embodiment, the priming mechanism can be displaced in the forward-drive direction relative to the plunger rod. It effects the priming stroke relative to the plunger rod. Compared with priming mechanisms in which the plunger rod effects not only the dispensing stroke but also the priming stroke, no mechanically complicated setting mechanisms are necessary in order to set the priming stroke. In one embodiment, the priming mechanism in accordance with the present invention also forms another plunger rod, although it does not necessarily effect the dispensing stroke. The plunger rod effecting the dispensing stroke, on the other hand, does not have to assume a priming function and in particular does not have to be set for the priming stroke of the plunger, which is generally significantly shorter than the dispensing stroke.

The priming mechanism in accordance with one embodiment of the present invention is advantageous for injection devices, including auto-injectors, as well as for semi-automatic injection devices. In the case of fully automatic and also semi-automatic injection devices, the plunger rod is driven in the forward-drive direction by the elastic force of a dispensing spring, in some preferred embodiments a mechanical dispensing spring, and is held in a retained position against the force of the dispensing spring prior to triggering and is thus biased in the forward-drive direction.

In some preferred embodiments, a fully automatic or semi-automatic injection device has an injection cannula pointing in the forward-drive direction serving as an infusing part. In principle, however, the injection device may also be a pressure injector without an injection cannula. In the case of an auto-injector with an injection cannula, the injection cannula is displaceable in the forward-drive direction in order to be able to effect the piercing action with an injection stroke. This being the case, in some preferred embodiments, the injection cannula is axially secured to a container forming the reservoir, which is displaceable relative to the housing in the forward-drive direction. To effect the piecing action, a drive structure which is driven in the forward-drive direction by means of an injection spring is able to drive the container with it in the forward-drive direction due to its own movement. In alternative embodiments, the forward-drive movement of the container, i.e. the injection stroke, is effected via the static friction of the plunger. To this end, the dispensing spring may also simultaneously serve as an injection spring, whereby it drives the container via the plunger in a first phase of the plunger rod stroke due to the plunger static friction and then or to a certain extent also simultaneously drives the plunger in the forward-drive direction in the container.

The priming mechanism, which is displaceable in the forward-drive direction prior to effecting the dispensing stroke or the injection and dispensing stroke relative to the plunger rod, offers advantages for injection devices of all designs and advantageously requires no adaptation imposed by the type of injection device due to the fact that its priming stroke can be effected independently of the plunger rod.

In some embodiments, one of the priming mechanism and plunger rod advantageously extends through the other in the forward-drive direction. In some embodiments, the plunger rod is sleeve-shaped and surrounds the rod-shaped priming mechanism. If the plunger rod is biased by a dispensing spring in order to effect the dispensing stroke, the dispensing spring is advantageously disposed in an annular gap between the priming mechanism and the plunger rod, at least across a major part of its axial length.

The device advantageously enables a product dose which can be dispensed during a dispensing stroke to be set, and has a dose metering element for this purpose. The dose metering element is disposed so that it can be displaced and is coupled with the plunger rod by means of a dose metering engagement. In some embodiments, the coupling is such that a dose metering displacement of the dose metering element results in a change in an axial distance of the plunger rod from the plunger prior to effecting a dispensing stroke. In alternative embodiments, the dose metering displacement of the dose metering element results in a change in the axial length of the dispensing stroke, which is expediently achieved by displacing a dispensing stop which restricts the dispensing stroke in the forward-drive direction.

The dose metering element may assume a dual function, in which case it forms an operating element for the priming mechanism as well. In some preferred embodiments, the dose metering element is advantageously disposed on the housing so as to be displaceable in the forward-drive direction. A dose metering and operating element displaceable in the forward-drive direction may be connected to the priming mechanism so that it drives the priming mechanism with it as it moves in the forward-drive direction, applying pressure in the forward-drive direction. This being the case, the dose metering and operating element also effects the priming stroke.

In some preferred embodiments, the priming mechanism couples the dose metering element with the plunger rod, i.e., the priming mechanism transmits the dose metering movement to the plunger rod. To this end, the priming mechanism may be connected to the dose metering element so that the dose metering element drives the priming mechanism with it during its dose metering movement, i.e., is rigidly connected to the priming mechanism for the dose metering movement. The coupling between the priming mechanism and the plunger rod may advantageously also be rigid during the dose metering movement. In some preferred embodiments, with regard to the dose metering movement, both the coupling between the dose metering element and the priming mechanism and the coupling between the priming mechanism and the plunger rod are rigid.

If the device enables the product dose to be set, it may advantageously also have a dose display. The dose display in turn has at least two display elements, one of which constitutes a dose scale, whilst the other forms a pointer for the dose display, in which case its position on the dose scale indicates the set product dose. One of the display elements is coupled with the priming mechanism so that the position of the pointer on the dose scale changes as the priming mechanism is displaced in the forward-drive direction. The change in position is advantageously such that once a priming stroke has been effected, if one is effected as a means of setting the product dose, the dose which can still be dispensed and, in some preferred embodiments, the dose which can be dispensed with the next dispensing stroke is displayed.

In some preferred embodiments, the priming mechanism transmits the dose metering movement to the plunger rod and is coupled with one of the display elements so that the displacement of the priming mechanism caused by the dose metering movement of the dose metering element causes a change in the dose display. In some preferred embodiments, the priming mechanism is not coupled with the dispensing movement of the plunger rod. In principle, however, it would be conceivable for the priming mechanism to effect the dispensing movement with the plunger rod as well but to be displaceable relative to the plunger rod in order to effect the priming stroke.

By virtue of another aspect of the present invention, an auto-injector comprises a housing, a product container accommodated by the housing, a plunger accommodated in the product container which is axially displaceable in the forward drive direction, a plunger rod and a dispensing spring. The dispensing spring applies a force to the plunger rod acting in the forward drive direction. The plunger rod is locked in a retained position in a releasable retaining engagement against the force of the dispensing spring. Ultimately, the locking action exists relative to the housing and may even be a retaining engagement directly between the plunger rod and the housing. However, in some preferred embodiments, the plunger rod is locked on a structure which is likewise axially displaceable, which is in turn in a retained position in a releasable retaining engagement with the housing. When the retaining engagement of the plunger rod is released, the plunger rod is driven by the force of the dispensing spring and effects a dispensing stroke, by means of which the plunger is moved in the forward direction in the container and dispenses a pre-defined product dose.

In some embodiments, the auto-injector is fitted with a displaceable dose metering element connected to the housing and the dose metering element is coupled with the plunger rod. The dose metering element can be operated and can effect a dose metering movement. The coupling with the plunger rod is such that the dose metering movement of the dose metering element enables a product dose to be set which can be dispensed by a dispensing stroke of the plunger rod. Consequently, an auto-injector is provided which permits the individual setting of a product dose that will be administered during the injection, not just by the manufacturer but also by a doctor, clinically trained staff person, the person to whom the product dose is administered, or a person self-administering the product dose. The coupling comprises a metering engagement based on a positive connection, in which the dose metering movement causes an axial displacement.

In some preferred embodiments, the plunger rod is not connected to the plunger but pushes against the plunger rear face in the forward direction by means of a front end as the product is being administered. In this embodiment, but also in embodiments in which the plunger rod is connected to the plunger, the product dose can be set due to the fact that a stop restricting the dispensing stroke in the forward drive direction is axially displaced by the dose metering movement of the dose metering element. In some preferred embodiments, the dispensing stroke is always of the same length irrespective of the set product dose, which can be achieved in the case of a plunger which merely pushes loosely against the plunger rod due to the shift in the axial distance between the plunger rod and the plunger. In such embodiments, the retaining position of the plunger rod can be axially shifted, for example.

In some preferred embodiments the plunger rod itself is length-adjustable and the dose metering movement of the dose metering element effects the adjustment in length. To enable its length to be adjusted, the plunger rod comprises several parts and of the several plunger rod parts, at least two are in a mutual dose metering engagement in which they are axially displaced relative to one another. A first one of the plunger rod parts comes into contract with the plunger in order to dispense the product. Another one of the plunger rod parts is in the retaining engagement in the position retaining the plunger rod. One of the plunger rod parts can be turned relative to the other in the dose metering engagement, e.g., about an axis pointing in the forward drive direction, so that the dose metering engagement converts the rotating movement into a relative axial movement. Not least because of its simplicity but also due to the possibility of achieving a continuous longitudinal displacement, a threaded engagement is one preferred option for the dose metering engagement.

The various parts of the plunger rod are connected to one another so that they effect the dispensing stroke jointly. In some embodiments, over and above the length adjustment, the dose metering engagement may also cause the plunger rod parts forming the dose metering engagement to drive each other along during the dispensing stroke so that they jointly effect the dispensing stroke.

In one embodiment, the plunger rod consists of two of such plunger rod parts, which sit in a dose metering engagement with one another. In a second embodiment, the plunger rod comprises more than two plunger rod parts, two of which sit in the dose metering engagement. Under certain circumstances, the various plunger rod parts may sit in a dose metering engagement with one another in respective pairs. In a preferred embodiment with three plunger rod parts or optionally even more plunger rod parts, a first one of the plunger rod parts is axially displaceable in the retaining engagement, a second one of the plunger rod parts sits in the retaining engagement and a third one forms the coupling or a part of the coupling between the dose metering element and the axially displaceable first plunger rod part. In some preferred embodiments, the third plunger rod part is not axially displaced by the dose metering movement.

In some preferred embodiments, the dose metering movement of the dose metering element is a rotating movement relative to the housing, e.g., the dose metering element effects the rotating movement about an axis of rotation pointing in the forward drive direction. In preferred embodiments wherein the plunger rod is longitudinally displaceable, one of its plunger rod parts is also displaceable in a rotating movement relative to another plunger rod part, and the axis of rotation of the dose metering element and the rotatably displaceable plunger rod part are flush. In one arrangement, the dose metering element and the rotatably displaceable plunger rod part can be connected to one another on the same axis of rotation when the plunger is in the retaining position and are unable to rotate.

In some preferred embodiments, the first plunger rod part is coupled with the dose metering element so that it is displaced relative to the second plunger rod part disposed in the retaining engagement due to its dose metering movement. It would also be possible to displace the second plunger rod part by means of the coupling with the dose metering element in the retaining engagement so that in the dose metering engagement either the axial position of the second plunger rod part shifts without releasing the retaining engagement or the axial position of the first plunger rod part is shifted.

In some preferred embodiments, the first plunger rod part extends through the second plunger rod part, in other words the second one surrounds the first one, at least across an axial portion of the length. The dispensing spring is accommodated in an annular gap remaining between the plunger rod parts. In the forward drive direction, it is supported directly on the second plunger rod part or pushes said third plunger rod part against the second.

During the dispensing stroke, the second plunger rod part which is in the retaining engagement in the retaining position is also moved into the container. In the setting in which the plunger rod is at its shortest length, the plunger rod parts abut axially.

In some preferred embodiments, an auto-injector in accordance with the present invention may be equipped with a dose display. The dose display comprises two display elements forming a dose scale and a pointer, the position of which indicates the product dose set on the dose scale.

In some embodiments, a sleeve body surrounding the pointer forms the dose scale and is transparent so that the position of the pointer is visible, at least in the region of the dose scale. The display element forming the dose scale may be a dose metering button disposed at a proximal end of the housing and constituting the dose metering element of the dose setting.

In the situation where the dose is set by means of a longitudinally displaceable plunger rod, the pointer of the dose display may be connected directly to the plunger rod part which is axially displaced in order to set the dose. The connection to the relevant plunger rod part is such that the pointer also effects the dose setting movement with the relevant plunger rod part. During the dispensing stroke, the connection should automatically release and the pointer should remain in the region of the dose scale. In another preferred embodiment, the pointer is uncoupled from the axial displacement of the axially displaceable plunger rod part or optionally of an axially displaceable plunger rod stop, but is coupled with the dose metering movement which causes the axial displacement. The coupling comprises a gear mechanism, e.g. a reducing gear, which translates or reduces the axial setting movement of the plunger rod or a plunger rod stop into an axial displacement of the pointer, in some preferred embodiments on a proportional basis. If the dose metering engagement is a threaded engagement, the reducing gear may also be a threaded engagement, with a thread which has a different, smaller pitch than the thread of the dose metering engagement.

In some preferred embodiments, one of the two display elements is disposed in axial alignment with the plunger rod and, as explained, may even sit directly on the plunger rod. The two display elements are disposed in alignment with the plunger rod. A bar transmitting the dose metering movement of the dose metering element to the plunger rod may be disposed in threaded engagement with the relevant display element, e.g., the pointer.

It should be appreciated that any of the embodiments, features, functions and/or structures described herein may be used cooperatively and/or to complement each other.

### BRIEF DESCRIPTION OF THE DRAWINGS

FIG. 1 shows an exemplary embodiment of an injection device with a variable dose in the “maximum dose” setting,

FIG. 2 illustrates the injection device illustrated in FIG. 1 in the “minimum dose” setting,

FIG. 3 illustrates the injection device shown in FIG. 2 with the injection cannula pushed forwards but before dispensing a product,

FIG. 4 illustrates the injection device shown in FIG. 2 with the injection cannula pushed forwards and after dispensing the product,

FIG. 5 illustrates the injection device of FIG. 1 on a larger scale,

FIG. 6 illustrates an embodiment of an auto-injector with a variable dose,

FIG. 7 illustrates another embodiment of an auto-injector with a variable dose, which is additionally equipped with a priming mechanism,

FIG. 8 illustrates another embodiment of an auto-injector with a variable dose in the “minimum dose” setting and with a priming mechanism,

FIG. 9 shows the auto-injector of FIG. 8 after priming,

FIG. 10 shows the auto-injector of FIG. 8 in the “maximum dose” setting, and

FIG. 11 shows FIG. 10 on a larger scale.

### DETAILED DESCRIPTION OF THE DRAWINGS

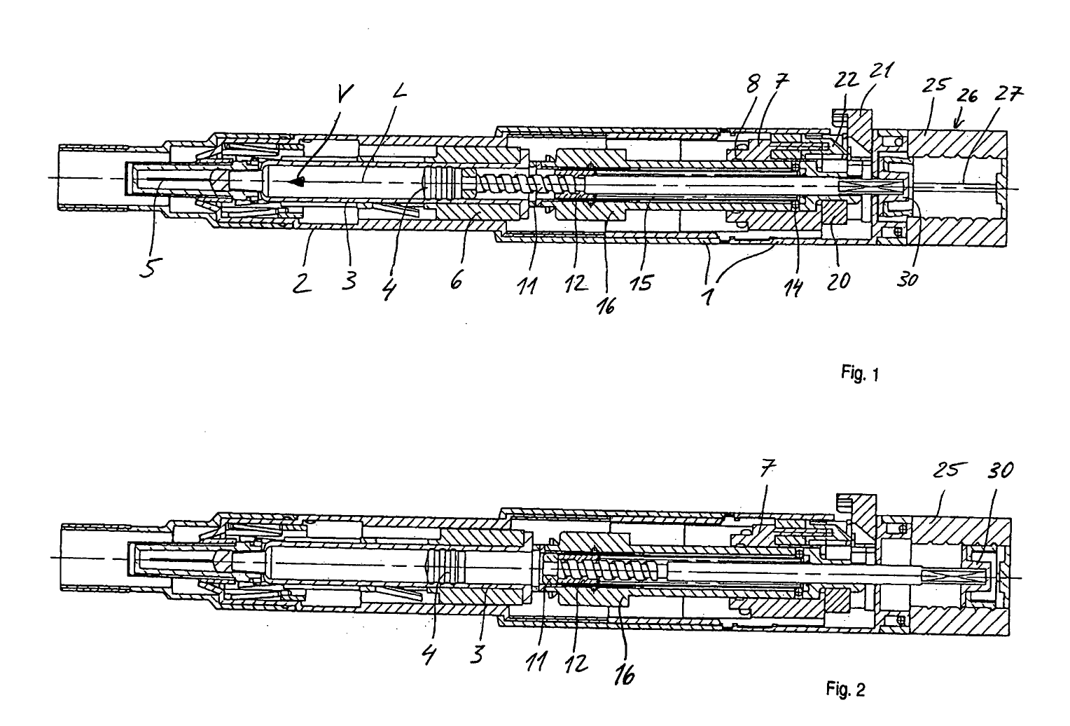

FIG. 1 illustrates an injection device with a variable dose, i.e., an auto-injector on which the desired dose of a product to be administered for the injection can be set by the user. The product may be a liquid medicament, for example insulin, a growth hormone or an osteoporosis preparation. FIG. 1 illustrates the injector in a setting corresponding to a maximum dose, i.e. the maximum product dose which can be administered by the injector will be dispensed on triggering.

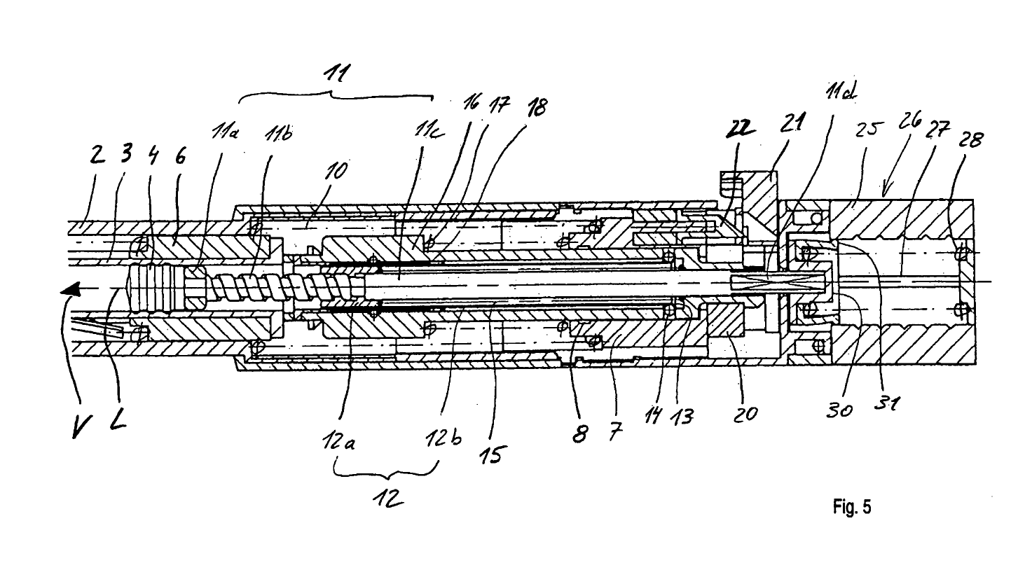

FIG. 5 illustrates a part of the injector on a larger scale and gives reference numbers for the components of the injector, whereas these are omitted in FIG. 1 in order to retain clarity. For the description of the first exemplary embodiment, reference should therefore be made to FIG. 5.

The injector has a housing, essentially comprising a first housing sleeve 1 and a second housing sleeve 2, which is displaceable relative to the first housing sleeve 1 along a common, central longitudinal axis L. The first housing sleeve 1 forms an axial slide guide for the second housing sleeve 2. Accommodated in the housing sleeve 2 is a container 3 filled with the product, which is displaceable in a forward-drive direction V along the longitudinal axis L. Attached to the front, distal end of the container 3 by reference to the forward-drive direction V is an injection cannula 5 pointing in the forward-drive direction V along the longitudinal axis L. A plunger 4 adjoins the container 3 at its rear, proximal end by reference to the forward-drive direction forming a seal. The plunger 4 is displaceable in the forward-drive direction V towards an outlet of the container, in order to eject the product out of the container 3 by means of a dispensing movement and dispense it through the injection cannula 5. The displaceable mounting of the container 3 is achieved by means of a container holder 6, into which the container 3 is pushed in the forward-drive direction V until it sits against a stop. The housing sleeve 2 forms an axial linear guide for the container holder 6 and hence for the container 3. The housing sleeve 2 not only surrounds the container holder 6 incorporating the container 3 but also the injection cannula as far as and beyond its tip. The housing sleeve 2 forms a needle guard.

A plunger rod causes the plunger 4 to be driven forward. The plunger rod comprises two plunger rod parts 11 and 12, which sit in a dose metering engagement in which the plunger rod part 11 effects an axial displacement relative to the plunger rod part 12 when effecting a dose metering movement. The dose metering engagement is a positive engagement.

The first plunger rod part 11 is rod-shaped and has as ram 11a and adjoining it, an engagement portion in the form of a threaded portion 11b and finally a shaft 11c with a coupling portion 11d at the end. The plunger rod part 11 operates solely by applying contact pressure to the plunger 4 in the forward-drive direction V. There is no connection between the plunger rod part 11 and the plunger 4 other than the contact pressure.

The plunger rod part 11 extends through the plunger rod part 12. The plunger rod part 12 is formed by a retaining sleeve 12b and an engaging part 12a. The engaging part 12a is pressed into the sleeve 12b at its front end. The engaging part 12a sits in the dose-metering engagement with the plunger rod part 11 and, in the embodiment illustrated as an example, in a threaded engagement with the threaded portion 11b.

The plunger rod is mounted so as to be axially displaceable in the forward-drive direction V in a drive structure 16 and is linearly guided by the drive structure 16 during such a movement, the dispensing movement. A distal linear guide is provided by means of a sleeve pressed into the drive structure 16, which guides the plunger rod part 12 in a sliding action and forms a stop 18, which restricts the dispensing movement of the plunger rod in the forward-drive direction V. A proximal linear guide is formed between the drive structure 16 and the plunger rod part 11. Accommodated in an annular gap between the plunger rod part 11 and the plunger rod part 12 is a dispensing spring 15, which in the embodiment illustrated as an example is a helical spring, which is supported on the drive structure 16 opposite the forward-drive direction V and on the plunger rod part 12, effectively its engaging part 12a, in the forward-drive direction, and biases the plunger rod with a spring force. The plunger rod is retained or blocked against the force of the biased dispensing spring 15 into a proximal retained position, in which the plunger rod part 12 sits in a releasable retained engagement with the drive structure 16. To achieve the retained engagement, the sleeve 12b widens outwardly at its proximal end, thereby forming a retaining shoulder 13 which is inclined at an angle with respect to the longitudinal axis L. The retaining shoulder 13 engages behind several blocking elements 14, which are guided in orifices of the drive structure 16 so that they can move transversely to the longitudinal axis L. The blocking elements 14 are cylindrical pins but could also be provided in the form of ball bearings, for example. The blocking elements 14 are biased radially outwards by the retaining shoulder 13 with a pressing force. When the plunger rod is in the retained position, the blocking elements 14 are prevented from flexing by means of a bearing block 7, which guides the drive structure 16 in an axial displacement and in which the blocking elements 14 surround the receiving axial portion. The bearing block 7 is fixedly joined to the housing 1.

An injection spring 17 biases the drive structure 16 in the forward-drive direction V against the container 3. The drive structure 16 is blocked in a retaining engagement in a proximal retained position, which is likewise releasable. In the retaining engagement, the drive structure 16 engages behind a blocking element 20. The blocking element 20 can be moved transversely to the longitudinal axis L out of the retained engagement with the drive structure 16 by operating a trigger element 21 via a transmission piece 22. The trigger element 21 is a trigger knob, which projects out of the housing sleeve 1 and is simply pushed inwards for triggering purposes.

The second plunger rod part 12 is mounted by means of the drive structure 16 and the bearing block 7 so that it can not rotate relative to the housing sleeve 1.

The drive unit, in some embodiments comprising the drive structure 16, the plunger rod and the two springs 15 and 17, is in the loaded state in which the drive structure 16 is blocked in its proximal retained position with respect to the housing sleeve 1, and the plunger rod is blocked in its proximal retained positioned with respect to the drive structure 16. In this state, the product dose which will be dispensed when the drive unit is triggered can be set. In order to set a product dose, the axial position of the plunger rod part 11 is adjusted relative to the axially fixed plunger rod part 12. As a result of the adjustment, the axial clearance distance between the plunger and the ram 11a is changed. The axial length of the dispensing stroke of the plunger rod is pre-determined by the proximal retained position of the plunger rod and the stop 18 and is not affected by the adjustment. Instead, the adjustment of the axial clearance distance within the dispensing stroke pre-determines the travel length across which the plunger rod part 11 pushes against the plunger 4 and thus drives it forward.

In FIG. 1, the plunger rod part 11 has assumed its most distal position relative to the plunger rod part 12, in which it is has the minimum distance, which may be “zero”, when the plunger rod is in the retained position relative to the plunger 4. In this setting, the plunger rod and the plunger 4 cover the biggest common travel length during a dispensing stroke and the maximum settable product dose, i.e. the maximum dose, is dispensed.

FIG. 2 illustrates the injector, also in the loaded state, but in the “minimum dose” position. The plunger rod part 11 has assumed its most proximal position relative to the plunger rod part 12, in which the ram 11a is in abutting contact with the end face of the plunger rod part 12a. The axial clearance distance between the plunger 4 and the plunger rod is at its maximum in the retained position and the travel length across which the plunger rod pushes against the plunger 4 during the course of a dispensing stoke is at its minimum.

In some embodiments, the injector is designed for one use only and is disposed of after the injection. It is given to the user, for example a person who will administer the product himself, or to doctors, in the loaded state with the container 3 holding the maximum dose. The user sets the desired product dose by setting the length of the plunger rod itself and in this sense individually.

The setting is made by means of a dose metering element 25, which is able to effect a dose metering movement relative to the housing sleeve 1. In the embodiment illustrated as an example, the dose metering element 25 is secured to the housing sleeve 1 so that it can effect a rotating movement about the longitudinal axis L, i.e., the dose metering movement is a rotating movement about the longitudinal axis L. In order to transmit the rotating movement, the dose metering element 25 is coupled with the plunger rod by means of a coupling element 27 so that it does not rotate about the longitudinal axis L. However, the plunger rod part 11 is able to move axially relative to the coupling element 27. The coupling element 27 is connected to the dose metering element 25 so that it does not rotate. In the embodiment illustrated as an example, it is integral with the dose metering element 25 in the form of a longitudinal rib of the dose metering element 25.

The dose metering element 25 is provided with a dose scale 26 in its sleeve part, in which dosage units are shown on an increasing scale from the proximal to the distal end. With its dose scale 26, the dose metering element 25 constitutes a first display element of a dose display. A second display element 30 of the dose display is forced onto the proximal end of the plunger rod part 11 by means of a spring 28 and is provided in the form of a hat-shaped shoulder. The second display element 30 forms a pointer 31, the axial position of which can be read on the dose scale 26. To this end, the dose metering element 25 is transparent in the region of the dose scale 26 or entirely transparent so that the pointer 31 is visible through the sleeve part of the dose metering element 25.

The coupling element 27 engages in a longitudinal groove of the display element 30, as a result of which the display elements 25 and 30 are prevented from rotating about the longitudinal axis but are connected so as to be axially displaceable relative to one another. The display element 30 sits on the coupling portion 11d of the plunger rod part 11 so that it can not rotate. As a result, the dose metering element 25 is connected to the plunger rod part 11 in a rigid rotating arrangement due to the coupling via 27 and 30.

The procedure involved in administering a product may be appreciated by observing the positions of components and the sequence of positions of components depicted in FIGS. 1 to 4.

In the loaded state, the product dose to be administered is set by turning the dose metering element 25, and in principle, every product dose between the maximum dose, such as administered with the setting illustrated in FIG. 1, and the minimum dose, such as administered with the setting illustrated in FIG. 2, may be selected. However, as it slides along the casing internal surface of the dose metering element 25, the pointer 31 latches in several discrete catch positions, which is perceptible to the user via a clicking noise, by touch or by other suitable means. The fact that the product dose is set in discrete steps, in this instance by means of the several catch positions of the pointer 31 which can be selected, increases the certainty that the desired product dose has actually been set.

Since the second display element 30 also effects the axial setting movement of the plunger rod part 11, the position of the plunger rod part 11 is displayed directly on the scale 26.

Once the product dose has been set or also beforehand, a cannula cap enclosing the injection cannula 5 is removed and pulled out of the housing sleeve 2. The injector is now ready for an injection. For the injection, the injector is gripped in the region of the housing sleeve 1 and placed vertically on the skin with its distal end at the injection site. The housing sleeve 1 is then pushed in the forward-drive direction, as a result of which it moves in the forward-drive direction V against the force of a spring 10 relative to the housing sleeve 2. During this relative movement, as the housing sleeve 2 moves in, it pushes the transmission piece 22 into an axial overlap with the trigger element 21 until it reaches an axial stop. The axial stop restricts the relative movement between the housing sleeves 1 and 2. The injection cannula 5 is now projecting almost as far as the distal end of the housing sleeve 2.

In the next step, the drive unit is triggered by depressing the trigger element 21. The trigger element 21 pushes radially against the transmission piece 22, which is radially displaceable in the axial position it has reached, and in turn pushes against the blocking element 20. Due to the fact that the trigger element 21 is depressed, the blocking element 20 is forced out of the retaining engagement with the drive structure 16. As soon as the retaining engagement is released, the injection spring 17 drives the drive structure 16 in the forward-drive direction V. The drive structure 16 is intrinsically axially rigid and due to its own forward movement forces the container holder 6 with the container 2 accommodated in it in the forward-drive direction V. The injection cannula 5 pierces into and through the skin as a result of this forward drive movement in order to administer the product subcutaneously.

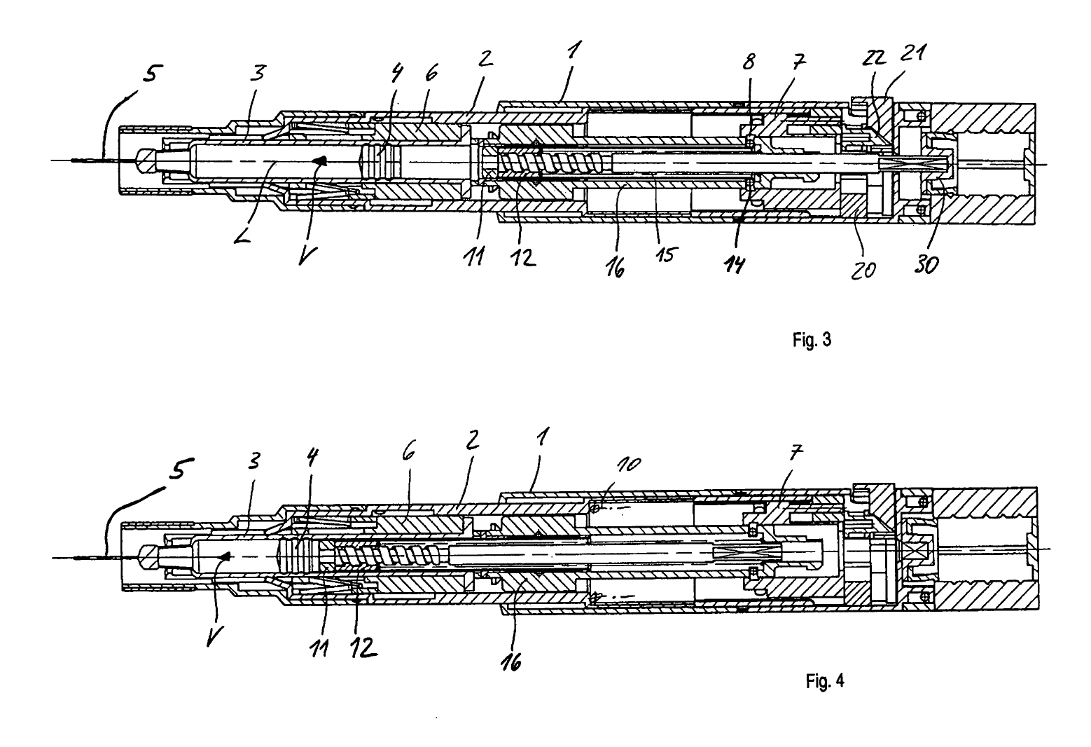

FIG. 3 illustrates the injector in the state in which the forward driving movement of the container 2 and hence the injection movement of the injection cannula 5 has just terminated but the dispensing stroke of the plunger rod has not yet started. In this state, after the injection phase and before the dispensing phase, the orifices of the drive structure 16 in which the blocking elements 14 are radially guided have reached a point overlapping with recesses 8 or a single circumferentially extending recess 8 formed in the casing internal surface of the bearing block 7 surrounding the drive structure 16. Due to the fact that they are now able to flex and because of the inclined contour of the retaining shoulder 13 or the plurality of retaining shoulders 13, the blocking elements 14 are forced outwards along the retaining shoulder 13 or retaining shoulders 13 so that the plunger rod part 12 is released from the blocking elements 14. The plunger rod part 12 moves in the forward-drive direction V due to the force of the dispensing spring 15 and because of the dose metering engagement drives the plunger rod part 11 with it. Once the plunger rod has covered the travel length from the plunger 4 corresponding to the set distance and is in pressing contact with the plunger 4, it forces the plunger 4 along the remaining course of the dispensing stroke in the container 2 in the forward-drive direction V, as a result of which the set product dose is dispensed through the injection cannula 5.

During the dispensing stroke, the second plunger part 12 also moves into the container 3. Its external dimensions in the cross-section are therefore restricted by the hollow cross-section of the container 3. In the case of slim containers, the second plunger rod part 12 may preferably be provided in the form of a metal sleeve 12b with an inserted engagement portion 12a because metal sleeve bodies can provide the same or even higher strength for a slimmer wall thickness than plastic sleeves.

During the course of the dispensing stroke, the display element 30 moves into abutment with a rear end face of the housing sleeve 1, so that the plunger rod is released from the display element 30.

FIG. 4 illustrates the injector after the set product dose has been administered but before the injection cannula 5 has been removed from the tissue. When the injector is pulled away from the injection site, the spring 10 pushes the housing sleeve 2 in the forward-drive direction V relative to the housing sleeve 1 until it reaches a stop position in which it surrounds the injection cannula 5 and extends beyond its tip. In its most distal position, the housing sleeve 2 is locked, ruling out injuries due to the injection cannula 5. The injector is then disposed of, for example returned to the manufacturer.

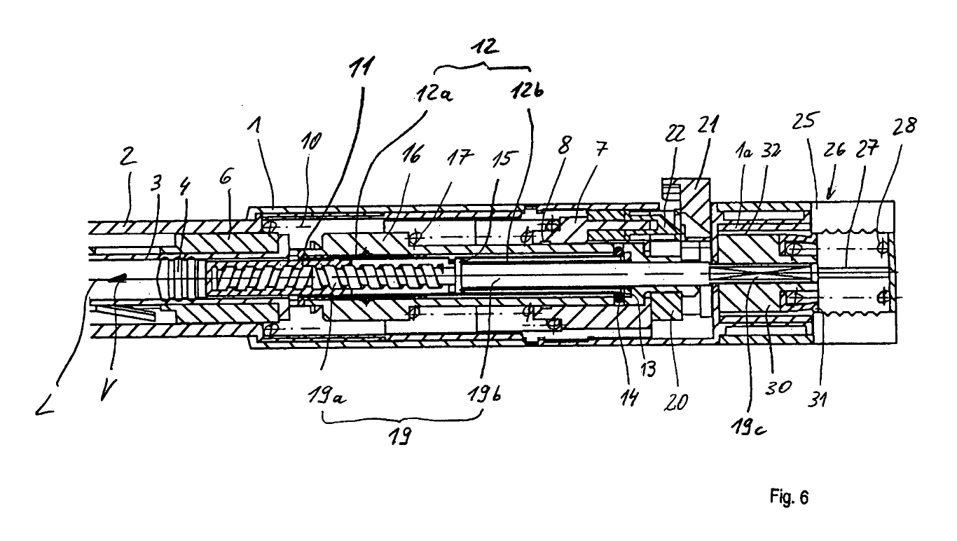

FIG. 6 illustrates a proximal or rear part of an auto-injector with a variable dose feature, based on a second exemplary embodiment. The injector is illustrated in the “maximum dose” setting. Components which fulfil the same function as those used in the initially described embodiment are denoted by the same reference numbers. Unless otherwise stated, the explanations given with respect to the initial embodiment apply to the second.

The plunger rod of the second exemplary embodiment is in three parts and consists of a first plunger rod part 11, a second plunger rod part 12 and a third plunger rod part 19. The first plunger rod part 11 again constitutes the ram and an engaging portion for the dose metering engagement. The first plunger rod part 11 is a sleeve with the engaging portion on its casing internal surface and the ram at its proximal end is provided in the form of a flat base. The engaging portion is again provided in the form of a threaded portion. The second plunger portion 12 is a cylindrical sleeve and again also has an engaging portion 12a and a retaining sleeve 12b with the retaining shoulder 13 at its proximal end. The plunger rod part 12 surrounds the first plunger rod part 11 and guides it linearly in the axial direction by means of its engaging portion 12a without rotating. The plunger rod part 12 is in turn non-rotatingly accommodated in the drive structure 16, which is in turn guided by the bearing block 7 and prevented from rotating. The third plunger rod part 19 consists of a distal engaging portion 19a, which establishes the dose metering engagement with the first plunger rod part 11 and is a threaded portion, and a proximal shaft 19b which axially guides the drive structure 16 and the end of which is provided in the from of a coupling portion 19c. The third plunger rod part 19 is rotatable about the longitudinal axis L. It is biased in the forward-drive direction V by the dispensing spring 15, and the dispensing spring 15 is supported on an outwardly projecting shoulder of the third plunger rod part 19. In the forward-drive direction, the shoulder abuts a complementary shoulder of the second plunger rod part 12, as a result of which the plunger rod is driven forwards when the injector is triggered. Accordingly, the dispensing spring 15 is ultimately supported on the second plunger rod part 12.

The third plunger rod part 19 projects out from the housing sleeve 1 beyond a rear end face and is connected to the dose metering element 25 via the display element 30 and the coupling element 27. Although the third plunger rod part 19 is able to rotate when the plunger rod is in the retained position, it is so tightly clamped between the dispensing spring 15 and the stop shoulder of the second plunger rod part 12 that it can be regarded as axially fixed to all intents and purposes when the plunger rod is in the retained position.

The second display element 30 is in a threaded engagement with the housing sleeve 1 about the longitudinal axis L. Its threaded portion is denoted by reference 32 and the threaded portion of the housing sleeve 1 by 1a. The display element 30, on the other hand, is connected to the plunger rod part 19 so that it can not rotate and is linearly guided in the axial direction. The product dose is again set by the dose metering rotating movement of the dose metering element 25 and transmitted to the third plunger rod part 19 by means of its coupling element 27 and the display element 30. The dose metering movement is transmitted, due to the dose metering engagement of the threaded portion 19a with the threaded portion of the first plunger rod part 11 and its axial linear guide, to an axial movement of the first plunger rod part 11. The display element 30 is axially displaced at the same time in its threaded engagement with the housing sleeve 1. The axial displacement of the display element 30 is reduced as a proportion of the axial displacement of the first plunger rod part 11 because a smaller thread is chosen for the threaded engagement of the display element 30 than for the threaded engagement of the first plunger rod part 11. This embodiment illustrates, in respect of the dose display, how the degree of axial displacement of the plunger rod, 19 can be increased or reduced in any way and transmitted to the axial displacement of the display element 30 and hence the pointer 31.

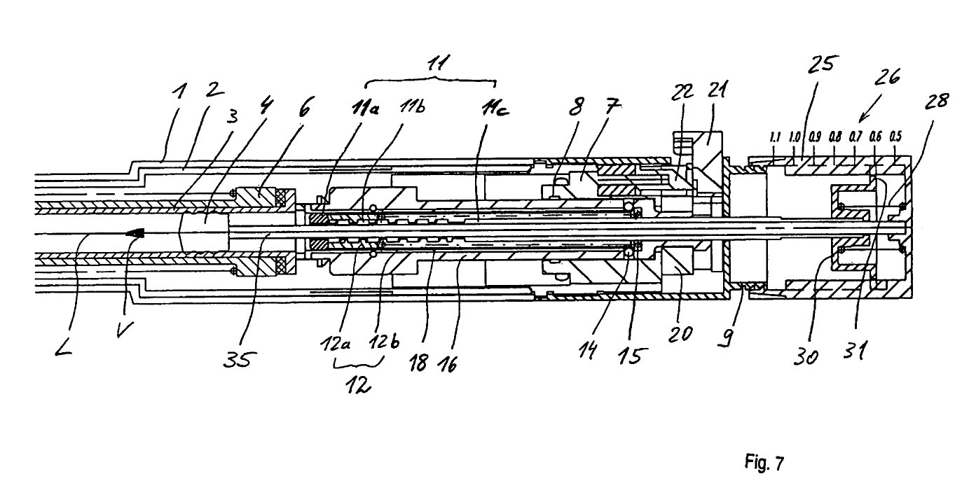

FIG. 7 illustrates a proximal or rear part of an auto-injector with a variable dose, in an exemplary third embodiment. The auto-injector is illustrated in the “minimum dose” setting. The same reference numbers as those used to refer to the initially described embodiment are used to denote components fulfilling the same function. In terms of its function, the drive unit corresponds to the drive unit initially described. In particular, the plunger rod again comprises two parts, consisting of the first plunger rod part 11 and the second plunger rod part 12.

Unlike the injectors of the first two exemplary embodiments, the auto-injector of the third embodiment is equipped with a priming mechanism 35, by means of which air can be removed from the components conveying the product as far as the tip of the injection cannula 5, although the latter is not illustrated, prior to the injection.

The priming mechanism 35 is a slim rod and extends though the plunger rod on the longitudinal axis L. Both plunger rod parts 11 and 12 surround the priming mechanism 35 concentrically. When the plunger rod is in the retained position, the priming mechanism 35 extends through the plunger rod, in the forward-drive direction V, so that it can act on the plunger 4 by means of its distal end in front of the plunger rod. However, it also extends through the plunger rod opposite the forward-drive direction V and is connected to the dose metering element 25 at its proximal end so that it is prevented from rotating about the longitudinal axis L and is not able to move axially. The plunger rod differs from the initially described plunger rod due to the fact that the first plunger rod part 11 of the third embodiment is sleeve-shaped to enable the priming mechanism 35 to extend through it.

The priming mechanism 35 is connected to the first plunger rod part 11 directly surrounding it so as to be prevented from rotating about the longitudinal axis L but is able to move in the forward-drive direction V relative to the first plunger rod part 11 and also relative to the other components of the drive unit. Conversely, the plunger rod part 11 is able to move axially relative to the priming mechanism 35 in order to set the product dose. The priming mechanism 35 is able to effect a priming stroke relative to the plunger rod and naturally also relative to the drive structure 16 in its respective retained position, during which it pushes against the plunger 4, driving it in the forward-drive direction V. The length of the priming stroke is dimensioned so that any air which might be left in the components carrying the product can be reliably forced out by the product but as little product as possible is dispensed for this purging process. The priming stroke is significantly shorter than the dispensing stroke.

The dose metering element 25 serves not only as a dose metering element for setting the product dose, but also as an operating element for the priming mechanism 35. To operate the priming mechanism 35, the dose metering element 25 is connected to the housing sleeve 1 so that it effects not only the dose metering movement relative to the housing sleeve 1, but also the priming stroke. At its proximal end, the housing sleeve 21 forms a connecting portion 9 for the connection to the dose metering element 25, which axially guides the dose metering element 25 and by means of which the metering element 25 is able to latch in several catch positions spaced at an axial distance apart from one another. The connecting portion 9 forms a catch portion with catch recesses and the dose metering element 25 has a matching catch mechanism, e.g., a catch lug 29. In the state as supplied, the dose metering element 25 is latched to the connecting portion 9 in the most proximal of the catch positions. From this catch position, it can be moved selectively in the forward-drive direction V into one of the other catch positions. An audible click indicates the latching action to the user. In this embodiment, 3 catch positions are provided in all. The dose metering element 25 can be rotated relative to the housing sleeve 1 from each catch position.

The dose display also takes account of the axial position of the priming mechanism 35, i.e., it takes account of whether a priming stroke has been effected and also the length of the priming stroke effected. To this end, the dose scale 26 is extended to include the maximum product dose which can be dispensed by one priming stroke. When a priming stroke is being effected, the display element 30 constituting the pointer 31, which is connected to the first plunger rod part 11 as in the initially described embodiment, remains axially stationary relative to the housing sleeve 1. During the priming stroke, however, the dose scale 26 is moved axially in the forward-drive direction V relative to the housing sleeve 1 and to the display element 30 so that the position of the pointer 31 on the dose scale 26 is changed accordingly. When the dose metering element 26 is moved due to axial pressure out of its most proximal catch position into its most distal catch position so that the priming mechanism 35 effects its biggest possible priming stroke, the dose metering element 25 and hence its dose scale 26 moves in the forward-drive direction V relative to the display element 30 and its pointer 31 and a residual dose remaining after the priming stroke has been effected is displayed. In this embodiment, half of the container contents can still be dispensed for an injection in the illustrated “minimum dose” setting after effecting the biggest possible priming stroke. In practical application, the product is set after the priming mechanism 35 has been operated, i.e. after effecting the priming stroke. However, the sequence can be reversed without any difficulty.

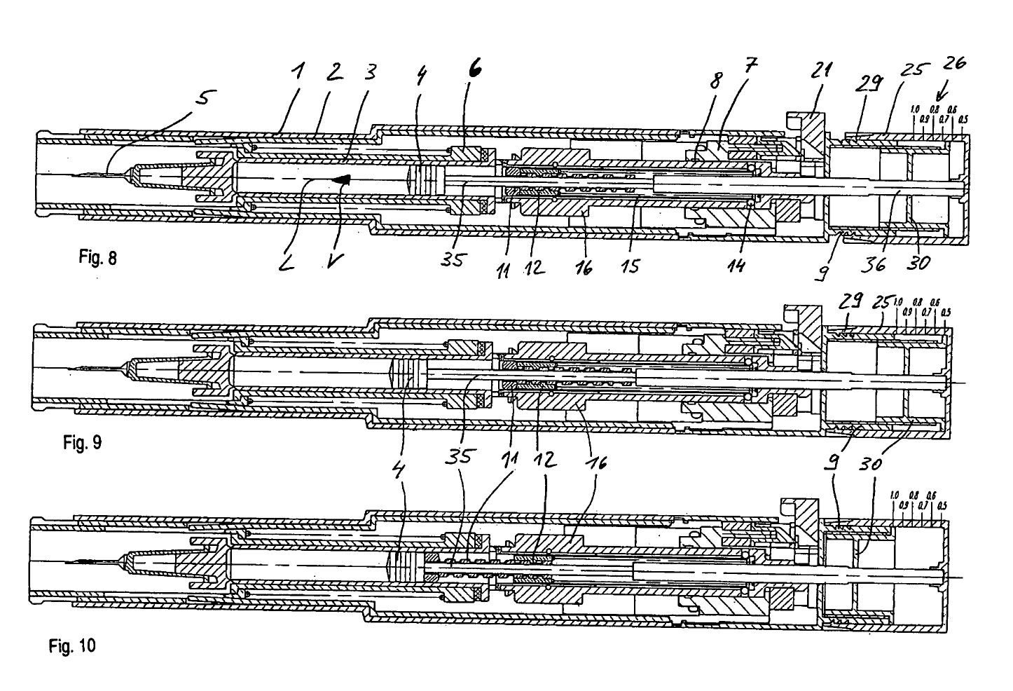

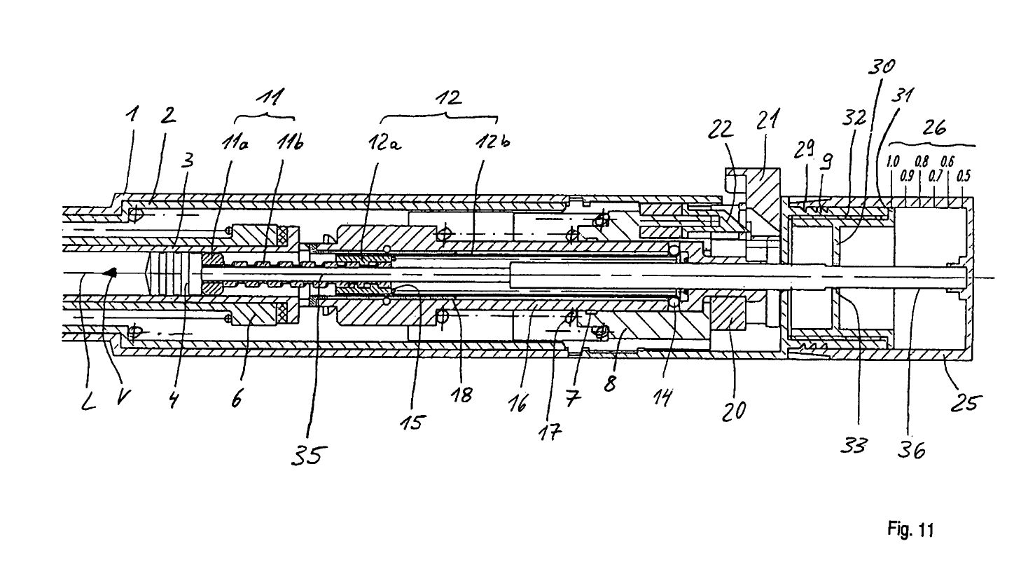

FIGS. 8 to 11 illustrate another embodiment of the present invention, auto-injector with a variable dose, which is also fitted with a priming mechanism 35. This embodiment differs from the third embodiment in terms of the priming mechanism 35 due to the fact that a dose display is provided in the same way as in the second embodiment, but with the priming mechanism 35. FIGS. 8 to 10 illustrate the injector with the priming mechanism 35 in different axial positions and the two extreme settings of the plunger rod. FIG. 11 illustrates the proximal part of the injector in the setting illustrated in FIG. 10 on a larger scale. In describing FIGS. 8 to 10, reference will also constantly be made to FIG. 11.

The plunger rod is again in two parts and the second plunger rod part 12 is the same as that of the first and the third embodiments. The first plunger part 11 consists of the ram 11a and the engaging portion 11b. Its axial length is that which is sufficient to displace it in the dose metering engagement with the second plunger rod part 12 when in its retained position from the setting for the minimum dose illustrated in FIG. 8 into the setting for the maximum dose illustrated in FIG. 10, in which it makes contact with the plunger 4. The second plunger rod part 12 yet again forms a stop, which restricts the axial displacement of the first plunger rod part 11 in and opposite the forward-drive direction V. The first plunger rod part 11 is a thin sleeve in its engagement portion 11b and, as in the third embodiment, is connected to the priming mechanism 35 so that it can not rotate but is axially displaceable. The priming mechanism 35 is in turn connected to the dose metering element 25 so that it can not rotate and therefore again forms the coupling between the dose metering element 25 and the plunger rod. Unlike the third embodiment, the display element 30 is axially displaceable relative to the priming mechanism 35 and is axially guided by the priming mechanism 35 but can not rotate.

The guide portion of the priming mechanism 35 is denoted by reference 36 and that of the display element 30 by 33. A threaded engagement with the housing sleeve 1 exists at 1a and 32. The dose display of the fourth embodiment therefore corresponds to that of the second embodiment (FIG. 6), in which the priming mechanism 35 fulfils the function of the third plunger rod part 19.

FIG. 8 illustrates the injector in a state in which the minimum dose which can be dispensed during the injection is set and the injector has not yet been primed. For priming purposes, the user pushes the dose metering element in the forward-drive direction V out of its illustrated most proximal catch position by means of the connecting portion 9 into one of the other catch positions. The priming mechanism 35 is moved in the forward-drive direction V by a travel path predefined by the catch positions as a result of this operation and also drives the plunger 4 in the forward-drive direction V so that any residual air in the system is forced out by the product.

FIG. 9 illustrates the injector set for the minimum dose and after the biggest possible priming stroke has been effected. In this state, the dose display indicates that during the injection, half of the maximum product dose which can be dispensed will be dispensed.

FIG. 10 illustrates the injector in the “maximum dose” setting and after effecting a biggest possible priming stroke. The dose display indicates that if the injector is triggered in this state, the maximum product dose which can be dispensed will be dispensed.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering an injectable product, comprising:
a) a housing.
b) a reservoir for the product,
c) a plunger axially displaceable in a forward-drive direction in the reservoir to effect a dispensing stroke in the forward-drive direction to dispense a product, and
d) a priming mechanism, displaceable in the forward-drive direction and acting on the plunger to permit a priming stroke in the forward-drive direction to remove air from the reservoir, wherein
e) the priming mechanism is moveable in the forward-drive direction relative to a plunger rod for the priming stroke.

2. The device as claimed in claim 1, wherein the priming mechanism is operable independently of the plunger rod.

3. The device as claimed in claim 1, wherein the priming mechanism is linearly guided in the forward-drive direction.

4. The device as claimed in claim 1, wherein one of the plunger rod and priming mechanism extends through the other in the forward-drive direction.

5. The device as claimed in claim 4, wherein the priming mechanism extends through the plunger rod.

6. The device as claimed in claim 1, the device further comprising a dose metering element, disposed on the housing so as to be displaceable and coupled by a dose metering engagement with the plunger rod so that a dose metering movement of the dose metering element causes a change in one of an axial distance of the plunger from the plunger rod or the axial length of the dispensing stroke.

7. The device as claimed in claim 6, wherein the dose metering element constitutes an operating element for the priming mechanism.

8. The device as claimed claim 7, wherein the dose metering element is disposed so as to be displaceable in the forward-drive direction and drives the priming mechanism with it as it moves in the forward-drive direction.

9. The device as claimed in claim 6, wherein the plunger rod comprises a first plunger rod part acting on the plunger and a second plunger rod part, and wherein one of the plunger rod parts is axially displaceable relative to the other.

10. The device as claimed in claim 9, wherein the plunger rod parts are in a dose metering engagement with one another.

11. The device as claimed in claim 9, wherein the plunger rod parts effect the same dose metering movement as the dose metering element.

12. The device as claimed in claim 6, wherein the priming mechanism couples the dose metering element with the plunger rod insofar as it transmits the dose metering movement to the plunger rod.

13. The device as claimed in claim 6, wherein the dose metering engagement is a threaded engagement about an axis extending in the forward-drive direction.

14. The device as claimed in claim 1, wherein the plunger rod has only a pressing contact with the plunger.

15. The device as claimed in claim 1, wherein the priming mechanism has only a pressing contact with the plunger.

16. The device as claimed in claim 1, further comprising a dispensing spring which acts on the plunger rod in the forward-drive direction, and wherein the plunger rod is blocked in a retained position against the force of the dispensing spring in a releasable retained engagement.

17. The device as claimed in claim 16, wherein the plunger rod comprises a first plunger rod part acting on the plunger and a second plunger rod part, and wherein one of the plunger rod parts is axially displaceable relative to the other and the second plunger rod part is blocked in the retained engagement.

18. The device as claimed in claim 16, wherein the device is an auto-injector.

19. The device as claimed in claim 18, further comprising:
an injection cannula pointing in the forward-drive direction and connected to the reservoir which is displaceable in the forward-drive direction, and
an axially displaceable structure, which carries the reservoir so that it can move in the forward-drive direction and is blocked in a retained position against the force of an injection spring in a releasable retaining engagement, wherein when the retaining engagement is released the reservoir and the injection cannula drive in the forward-drive direction.

20. The device as claimed in one claim 1, wherein the device has a dose display with at least two display elements, one of which forms a dose scale and the other a pointer of the dose display, and one of the display elements is coupled with the priming mechanism so that the position of the pointer is moved on the dose scale during a priming stroke.

21. The device as claimed in claim 20, wherein one of the display elements constitutes an operating element for the priming mechanism.

22. The device as claimed in claim 21, wherein the display element constituting the operating element is in a latched engagement with the housing which can be released by the priming stroke.

23. The device as claimed in claim 22, wherein at least one of the display elements is coupled with the plunger rod so that an axial displacement of the plunger rod causes a displacement of the coupled display element relative to the other display element.

24. The device as claimed in claim 23, wherein the display element coupled with the plunger rod effects the same axial displacement as the plunger rod.

25. The device as claimed in claim 23, wherein the plunger rod is coupled with at least one of the display elements by means of a gear mechanism.

26. The device as claimed in claim 25, wherein the priming mechanism couples the at least one of the display elements with the plunger rod.

27. The device as claimed in claim 20, wherein one of the display elements surrounds the other and is transparent at least in a region which forms or surrounds the dose scale.

28. The device as claimed in claim 20, the device further comprising a dose metering element, disposed on the housing so as to be displaceable and coupled by a dose metering engagement with the plunger rod so that a dose metering movement of the dose metering element causes a change in one of an axial distance of the plunger from the plunger rod or the axial length of the dispensing stroke, wherein the dose metering element forms one of the display elements.

29. An auto-injector comprising:
a) a housing,
b) a product container,
c) a plunger axially displaceable in a forward drive direction in the product container,
d) a plunger rod which effects a dispensing stroke in the forward drive direction in order to dispense a product, and
e) a dispensing spring acting on the plunger rod in the forward drive direction,
f) wherein the plunger rod is locked in a retaining position in a releasable retaining engagement against the force of the dispensing spring, wherein
g) a dose metering element is disposed on the housing so that it can be displaced in order to set a product dose and is coupled with the plunger rod by a dose metering engagement so that a dose metering movement of the dose metering element causes an axial displacement of one of the plunger rod or a stop for the plunger rod when the retaining engagement is in effect.

30. The auto-injector as claimed in claim 29, wherein the plunger rod has an adjustable length and comprises a first plunger rod part acting on the plunger and a second plunger rod part, and when the plunger rod is in the retaining engagement, the first plunger rod part is axially displaceable relative to the second plunger rod part in the dose metering engagement.

31. The auto-injector as claimed in claim 31, wherein the first plunger rod part and the second plunger rod part are in a dose metering engagement with one another which transmits the dose metering movement of the dose metering element transmitted to one of the plunger rod parts to the axial displacement of the first plunger rod part.

32. The auto-injector as claimed in claim 30, wherein the plunger rod has a third plunger rod part in the dose metering engagement with the first plunger rod part, which transmits the dose metering movement of the dose metering element transmitted to one of the plunger rod parts to the axial displacement of the first plunger rod part.

33. The auto-injector as claimed in claim 32, wherein the second plunger rod part forms a retaining shoulder with which it is in retaining engagement.

34. The auto-injector as claimed in claim 33, wherein the second plunger rod part has an engagement part at one axial end and the retaining shoulder at the other axial end.

35. The auto-injector as claimed in claim 34, wherein the second plunger rod part includes a retaining sleeve with an inner shoulder, and the dispensing spring extends into the retaining sleeve and acts on the shoulder.

36. The auto-injector as claimed in claim 35, wherein one of the plunger rod parts surrounds the other.

37. The auto-injector as claimed in claim 36, wherein the dispensing spring is disposed in an annular gap between the plunger rod parts.

38. The auto-injector as claimed in claim 29, wherein the dose metering engagement is a thread engagement.

39. The auto-injector as claimed in claim 29, further comprising an axially displaceable drive structure which drives the container in the forward drive direction during an injection and with which the plunger rod is in retaining engagement.

40. The auto-injector as claimed in claim 39, wherein the drive structure is releasably locked in a retaining position against a force of an injection spring acting in the forward drive direction.

41. The auto-injector as claimed in claim 29, wherein the dose metering element is disposed on the housing so as to be rotatable about an axis pointing in the forward drive direction.

42. The auto-injector as claimed in claim 29, wherein the dose metering element is coupled with the plunger rod so as to be prevented from turning.

43. The auto-injector as claimed in claim 29, wherein the dose metering element is disposed in an axially flush alignment with the plunger rod.

44. The auto-injector as claimed in claim 29, further comprising display elements, one of which forms a dose scale and the other a pointer, the position of which on the dose scale displays a product dose set by means of the axial displacement of the plunger rod.

45. The auto-injector as claimed in claim 44, wherein at least one of the display elements is coupled with the plunger rod so that the axial displacement of the plunger rod causes a displacement of the coupled display element relative to the other display element.

46. The auto-injector as claimed in claim 45, wherein the display element coupled with the plunger rod also performs the axial displacement of the plunger rod.

47. The auto-injector as claimed in claim 45, wherein the plunger rod is coupled with at least one of the display elements by means of a gear mechanism.

48. The auto-injector as claimed in claim 44, wherein one of the display elements surrounds the other and is transparent in at least a region forming or surrounding the dose scale.

49. The auto-injector as claimed in claim 44, wherein the dose metering element constitutes one of the display elements.
